(19) United States
(12) Patent Application Publication
(10) Pub. No.: US 2008/0175851 A1
(43) Pub. Date: Jul. 24, 2008

(54) METHODS AND COMPOSITIONS FOR ENHANCING THE GROWTH OF FISH AND SHELLFISH

(76) Inventors: Mingder Yang, Madison, WI (US); Bryan Renk, Sun Prairie, WI (US); Scott Schneider, Jefferson, WI (US); Carrie Cook, Verona, WI (US)

Correspondence Address: FOLEY & LARDNER LLP, 150 EAST GILMAN STREET, P.O. BOX 1497, MADISON, WI, 53701-1497, US

(21) Appl. No.: 11/954,113

(22) Filed: Dec. 11, 2007

Related U.S. Application Data
(60) Provisional application No. 60/886,447, filed on Jan. 24, 2007.

(51) Int. Cl. A61K 39/395; A61P 3/00

(52) U.S. Cl. 424/157.1

(57) ABSTRACT

Methods and compositions for enhancing weight gain and/or feed efficiency in fish and shellfish are disclosed. The methods include administering an agent, such as an antibody, which reduces the bioavailability in fish or shellfish of a prostaglandin or leukotriene lipid precursor. The methods also include administering an antibody which enhances weight gain by at least 10%, improves feeding efficiency by at least 5%, or both. Fish and shellfish food comprising antibodies as described herein are also disclosed.

### CROSS-REFERENCE TO RELATED APPLICATIONS

This application claims priority to U.S. provisional patent application No. 60/886,447, filed Jan. 24, 2007, the entire disclosure of which is incorporated herein by reference.

### FIELD OF THE INVENTION

The present invention relates generally to aquaculture and to methods and compositions that enhance the growth or improve the feed efficiency for fish and shellfish. In particular, there are provided antibodies, such as anti-PLA₂, anti-CD14 or anti-TLR4 antibodies, that may be administered to fish and shellfish to enhance their growth and improve feed efficiency.

### BACKGROUND OF THE INVENTION

Aquaculture involves the commercial fanning of fish and shellfish, typically for human consumption. Often the fish are kept in tanks or enclosures where their diet is supplemented or entirely derived from external sources of food. It is common practice to feed fish solid compositions comprising protein, fat, fiber, and minor amounts of adjuvants, such as minerals, vitamins, and trace elements. The rate at which food is converted to body protein is relatively low, and the protein component of the food must be chosen among relatively expensive sources to avoid damage to the fish (See Klaus Hoffman, “Immunostimulation in Aquaculture: What's the News?,” Aqua Culture Asia Pacific 3(5) (2007)). Consequently, it is desirable that commercially-raised fish and shellfish grow to a marketable size quickly, using the minimum amount of food.

### SUMMARY OF THE INVENTION

Methods for significantly enhancing growth and/or improving feed efficiency for fish and shellfish have been discovered. In one aspect, the methods include administering to a fish or shellfish an antibody that reduces the bioavailability of a prostaglandin or leukotriene lipid precursor The amount of antibody administered is effective to increase feed efficiency and/or weight gain compared to fish or shellfish which have not been administered the antibody. Suitable antibodies include, for example, anti-phospholipase A₂ (anti-PLA₂) anti-CD14 or anti-Toll 4 receptor antibody and the like. The weight gain of the fish or shellfish to which the antibody is administered may range, e.g., from about 1% to about 30%, to about 50% or even to about 100%. For example weight gain can be at least 5%, at least 10%, at least 15%, at least 20%, or at least 25% compared to fish or shellfish that have not been administered an antibody as described herein. Likewise, the increase in feed efficiency in fish or shellfish administered the antibody may range from about 1% to about 5%, to about 10%, to about 15%, or even to about 20%, and can be at least 5%, at least 6%, at least 7%, at least 8%, at least 9% or even at least 10% in comparison to the untreated fish or shellfish,

In another aspect of the invention, the methods include administering to a fish or a shellfish an antibody that enhances weight gain by at least 5%, improves feeding efficiency by at least 1%, or both, compared to untreated fish. In some embodiments the weight gain is enhanced by at least 10% and/or the increase in feed efficiency is at least 2%, at least 3%, at least 4% or at least 5% Suitable antibodies include anti-PLA₂, TLR4, CD14, and the like. In some embodiments, the antibody used is an anti-PLA₂ antibody. In other embodiments, the antibody is TLR4 and/or CD14 and is administered to a shellfish.

A wide variety of fish are suitable for use in methods described herein, including, but not limited to trout, tilapia, perch, catfish, salmon, bass, sunfish, and baitfish. Likewise, various shellfish may be used in methods described herein. Exemplary shellfish include, but are not limited to shrimp, prawns, mussels, clams, oysters, lobster, crab, and crayfish.

The antibody may be administered by any methods known to those skilled in the art and may be conveniently administered by feeding it to the fish or shellfish in food. In some embodiments, the food comprises an egg preparation that comprises the antibody.

In another aspect, the invention provides compositions containing an antibody of the invention. Thus, for example, there are provided compositions that include fish or shellfish food and an effective amount of an antibody that reduces the bioavailability of a prostagl andin or leukotriene lipid precursor, or an antibody that enhances weight gain by fish or shellfish by at least 10% or improve feeding efficiency of the fish or shellfish by at least 5% compared to fish or shellfish which have not been administered the antibody. Typically, the fish and shellfish food includes protein and fat. In some embodiments, the food includes by weight from 30% to 65% protein and from 5% to 25% fat. In other embodiments, the fish food includes by weight from 40% to 50% protein and from 10% to 20% fat. In certain embodiments, the fish food comprises an egg preparation that comprises the antibody, e.g., a powdered egg preparation is added in an amount from about 0.01% to about 10% by weight, from about 0.1 to about 5% by weight, from about 0.1% to about 3% by weight, from about 0.1% to about 2% by weight, from about 0.1% to about 1% by weight, or froim about 0.15% to about 1% by weight. The amount of added egg preparation in the feed will vary according to the concentration of antibody in the egg preparation and the particular application. It is well within the skill in the art to select the appropriate amount for a particular application in view of the guidance provided herein. In some embodiments, the fish food composition may be sprayed with an oil to reduce product separation such as leeching. For example, the oil may be a vegetable oil that is added to a level of from about 0.5 wt % to about 5 wt % such as about 0.5 wt %, about 1 wt %, about 2 wt %, or about 5 wt % of the fish food composition. In some embodiments, the oil is suitably added at about 2 wt % in the final ration.

An unexpected advantage of the present compositions and methods is the ability to substitute synthetic or plant-derived lipids and protein for fish oil and fish-meal protein in fish and shellfish food. Canola oil and soybean meal are the most familiar plant-derived substitutions used in commercial fish feed, although flaxseed, hempseed, and soy oils, and wheat, corn, barley, oats, canola, and sunflower meals are also employed (K. S. Betts, Environmental Science and Technology, Feb. 12, 2004). For example, fish food containing soybean meal can trigger stress in many species of fish resulting in suppressed appetite, weakened immune function, intestinal damage, and physical alterations which damage the market value (Overturf et al., Aqiaciilture America, p. 220 (2006)). Thus, fish food is typically formulated with large amounts olf fish meal as the primary protein source, with fish oil as the nutritional lipid source. In the present compositions, the addition of antibodies that reduce the bioavailability of prostaglandin or leukotriene lipid precursors allows the fish to better tolerate plant-derived food sources. Thus, compositions provided herein may include plant-derived proteins such as soybean-meal. In some embodiments, the amount of plant-derived protein ranges from about 1% to about 100% firom about 1% to about 75%, and preferably from about 1% to about 50%. The compositions provided herein may include plant-derived lipids, such as rapeseed oil, in addition to or as a replacement for fish oil (see Bell et al., Aquaculture 218: 515-52 (2003). In some embodiments. the amount of plant-derived lipids ranges from about 1% to about 100% from about 1% to about 75%, and suitably from about 50% to about 100%.

### BRIEF DESCRIPTION OF THE DRAWINGS

FIG. 1 is a graph showing the weight gain (gams per fish) over time of fish administered the antibodies of the invention.

### DETAILED DESCRIPTION

It has unexpectedly been discovered that the methods described herein result in a remarkable increase in weight gain and/or feed efficiency in fish and shellfish to which certain antibodies are administered. The improved weight gain and feed efficiency is surprising in view of the significant differences in the digestive and immune systems between fish shellfish and mammals from which the antibodies are derived. Although not wishing to be bound by theory, it is believed that the increase in weight gain and/or feed efficiency results from lowering gastric inflammation with antibodies that, e.g. reduce the bioavailability of a prostaglandin or leukotriene lipid precursor in the fish or shellfish or reduce formation of a complex on the surface of a cell in the gastrointestinal tract of the fish or shellfish, wherein the complex comprises endotoxin, TLR4 and/or CD14.

Antibodies described herein can be administered by injection or by oral delivery, and may be administered in combination with a suitable carrier of the type commonly used in delivery of pharmaceuticals or nutritional supplements. Injection methods include, but are not limited to, subcutaneous, intraperitoneal, intramuscular, or intravenous injection. Oral administration, can include, but is not limited to, administration in tablet or powder form. Most preferably, the agent is fed directly by mixing with feed or by coating feed particles as described in U.S. Pat. No. 5,725,873, incorporated herein by reference in its entirety.

In one embodiment, the antibodies may be administered in fish feed. Where the feed is provided to fish or shellfish having an aggressive feeding behavior, an egg powder comprising the anti-PLA2 antibody may be mixed directly with the fish feed. Because the fish consume the feed quickly, the antibody is unlikely to leech from the feed. However, in some other aquaculture species, e.g., yellow perch, shrimp, etc., feeding normally takes up to 30 minutes between the time feed is provided into the tanks and when the feed is completely consumed. This creates a potential problem, as the antibody may leech from the feed before it is consumed. Accordingly, in one embodiment, vegetable oil may be sprayed on the feed preparation. For example, a 2% solution of vegetable oil in water may be sprayed on the feed after the antibody powder has been mixed with the feed.

In one method, antibodies are prepared as follows. A producer animal is immunized with a peptide or protein, such as PLA₂, against which antibodies are desired so that the producer animal produces an antibody to said peptide or protein. A substance containing the antibody is obtained from said producer animal. The antibody can be subject to further purification if desired or can be used without further preparation in the fish or shellfish feed.

The method of Tokoro (U.S. Pat. No. 5,080,895), as exemplified in Cook (U.S. Pat. No. 6,383,485), can be used to produce a preparation of egg-yolk antibodies. Laying hens, e.g., can be inoculated with PLA₂. Preferably, a suitable adjuvant is administered in conjunction with the hen PLA₂ inoculation to enhance the immune response. An adjuvant useful for this purpose is a water-in-oil emulsion adjuvant such as complete Freund's adjuvant. The PLA₂ causes the hens to produce anti-PLA₂ antibodies which are passively transferred into the egg yolk of eggs laid by the hens. Typically, chicken eggs contain approximately 100-150 mg IgY, or roughly 2-3 mg/mL (Rose et al., Develop. Comp. Immunol. 5:115-20 (1981), 5-10%, or even up to 15% of which is specific for the inoculated antigen. Thus, a whole egg preparation can be expected to contain 10 mg of specific IgY.

An egg preparation, e.g., egg yolks or whole eggs, containing the anti-PLA₂ antibody can be collected and homogenized to form an emulsion. The resulting emulsion can be dried to form a powder containing the anti-PLA₂ antibody. This powder can then be formulated in a manner appropriate to the administration route and then administered to the desired fish or shellfish using methods known in the art. The preparation is preferably administered orally, e.g., as a supplement to the diet of the fish or shellfish. It is well within the skill in the art to adjust the amount of antibody administered to the fish or shellfish based on particular application, the results desired, and the guidance provided herein.

One or more additional fish food supplements may be combined with an anti-PLA₂ antibody to generate a fish food composition. In one embodiment, the fish food composition may further comprise probiotics (e.g., Bacillus, Lactobacillus, Enterococcus, Carnobacteriam, Saccharomyces, or Candida). The fish food composition may also comprise prebiotics (e.g., fructooligosaccharides, lipopolysaccharides, and glucans). In addition, the fish food composition may include supplements such as nucleic acids, nucleotides, metabolites, enzymes, antibiotics, or other egg antibody products.

In other embodiments, taurine, methionine, beta-glucans, hormones, or immunostimulants may be added to the fish food composition. Taurine, methionine, and beta-glucans have been used to boost growth in fish grown with diets high in plant materials (Gaylord et al., 2007. Aquaculture 269: 514-525; Sealey et al., 2006. Book of Abstracts Aquaculture America). Hormones have also been studied to increase growth and muscle mass in fish, although possibly this can only be effectively administered by injection (Simpson, et al., 2004. General and Comparative Endocrinology 135: 324-333). Additionally, various immunostimulants have been proposed as feed supplements (Galindop-Villegas, J. & H. Hosokawa. 2004. Immunostimulants: Towards Temporary Prevention of Diseases in Marine Fish In: Advances en Nutricion Acuicola VII Memorias del VII Simposium Internacional de Nutricion Acuicola, 16-19 Nov. 2004. Hermosillo, Sonora, Mexico) which could possibly have auxiliary benefits of increased growth.

All publications, patent applications, issued patents, and other documents referred to in this specification are herein incorporated by reference as if each individual publication, patent application, issued patent, or other document were specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

### Example 1

### Effect of Anti-PLA₂ on Trout

A blind study was conducted to test the effects of anti-PLA₂ (from aOva) on juvenile rainbow trout growth. The study included three treatment groups: control, 0.15% and 0.30% anti-PLA₂ powder produced as in U.S. Pat. No. 6,383,485. Each treatment group involved six 115-L tanks, each stocked with 12 fish (˜25 g initial weight) for a total of 18 tanks. The feed was extended Silver Cup steelhead diet (45% protein, 16% fat). The fish were carefully fed to apparent satiation once daily, and total feed input to each tank was recorded.

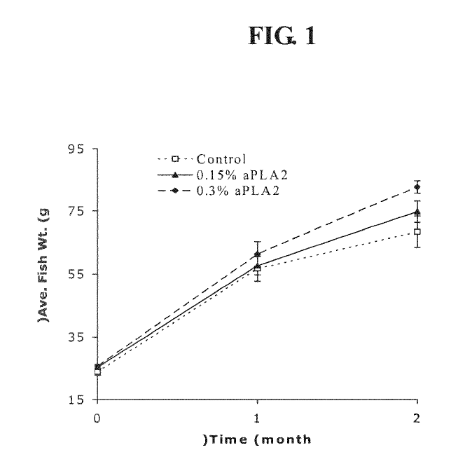

Results of the study are shown in Table 2. After 2 months, fish in the 0.15% and 0.3% treatment groups grew 11.1% (P=0.1), and 27.8% (P=0.03) greater than controls, respectively (Table 2, FIG. 1). Feed conversion ratios in the control, 0.15%, and 0.3% treatment groups were 0.93, 0.93 and 0.86, respectively (Table 2). These results show that anti-PLA₂ is a very effective stimulator of growth in rainbow trout, and that the potential for growth improvement in fish may be significantly greater than in terrestrial farm animals where growth improvements have been closer to 5-10%. As such, feed comprising the anti-PLA₂ antibody of the invention is useful in methods to improve feeding efficiency and/or weight gain in fish or shellfish administered the composition.

TABLE 2
Effects of aPLA₂ on rainbow trout growth.

| | | % Weight gain | | Final |
|---|---|---|---|---|
| Treatment | Weight gain (g) | relative to control | FCR | weight (g) |
| Control | 44.3 ± 4.2 | | 0.93 | 68.2 |
| 0.15% anti-PLA₂ | 49.3 ± 3.1 | 11.1% | 0.93 | 74.6 |
| 0.30% anti-PLA₂ | 56.6 ± 1.9* | 27.8% | 0.86 | 82.6* |

FCR = feed conversion ratio (feed consumed/weight gained).
*P < 0.05 relative to control.

### Example 2

### Effect of Anti-PLA₂ on Commercially Farmed Trout

A second study examined the effects of anti-PLA₂ on growth and feed efficiency in a commercial trout farming operation. The study included two treatment groups: control and 0.3% anti-PLA₂ powder produced as in U.S. Pat. No. 6,383,485. Each treatment group involved two hatchery tanks, each stocked with 3,500 fish (˜10 g initial weight) for a total of 4 tanks. The feed mixture was Silver Cup Steelhead feed and comprised 45% protein and 12% fat. The fish were fed twice a day to apparent satiation. Total feed input to each tank was recorded.

Results of the study are shown in Table 3. After 27 days, fish in the anti-PLA₂ treatment group exhibited a 174.6% change in body weight, while the control group exhibited a 179.9% change in body weight, Fish in the anti-PLA₂ tanks utilized significantly less feed compared to the controls. Consequently, fish that are administered anti-aPLA₂ feed compositions exhibited 16% feed savings in this study. Because trout feed is approximately $800 per ton, significant cost savings can be realized by using the claimed compositions.

TABLE 3
Effects of Anti-PLA₂ on Commercial Trout Feed Efficiency.

| | | % Body Weight - | |
|---|---|---|---|
| | Feed Intake | Relative Change: | Feed Savings - |
| Treatment | (lbs) | Day 1 to Day 27 | Relative to Control |
| Control | 125 | 179.9% | |
| anti-PLA₂ | 105 | 174.6% | 16% |

### Example 3

### Vegetable Oil Coating to Prevent Leeching

To investigate methods of preventing leeching of the antibody from the fish food, pellets were treated with vegetable oil. Briefly, 0.3% anti-PLA₂ powder was mixed with Silver Cup Steelhead Feed. After the egg antibody powder had been thoroughly mixed with the feed, the mixture was sprayed with 2% vegetable oil on the feed. This appears to cause the egg antibody powder to adhere to the fish feed pellets more tightly. Leeching of the antibody was reduced 87% after one hour exposure compared to untreated controls. Thus, spraying food/antibody mixtures with vegetable oil can be useful in aquaculture where the fish or shellfish do not exhibit aggressive feeding behavior.

### Comparative Examples

A comparative study of the effects of anti-PLA₂ on chick growth and feed efficiency was carried out essentially as described in U.S. Pat. No. 6,383,485. The three week study employed 16 groups of 10 chicks each split evenly between control (no anti-PLA₂) and treated (anti-PLA₂). Chicks which were fed anti-PLA₂ showed an average weight gain of 5.3% (P<0.1) over untreated chicks and an average increase of feed efficiency of 3.8% (P<0.05) compared to untreated chicks.

A comparative study of the effects of anti-PLA₂ on swine growth and feed efficiency was carried out similarly to above. The anti-PLA₂ treated swine exhibited an average weight gain of 5.5% and an average increase in feed efficiency of 3.6% over untreated swine.

The comparative examples show the remarkable effect of an antibody of the invention on fish. In chicken and swine studies, the average weight gain is about 5-6% and the increase in feed efficiency is about 3-4%. By comparison, administration of anti-PLA₂ at the highest doses to trout provided a remarkable 28% increase in weight gain and a nearly 8% gain in feed efficiency over untreated fish.

What is claimed is:

1. A composition comprising fish or shellfish food and an antibody that reduces the bioavailability of a prostaglandin or leukotriene lipid precursor, wherein the antibody is present in an effective amount for increasing weight gain or feed efficiency in fish and shellfish administered the antibody compared to fish or shellfish not administered the antibody.

2. The composition of claim 1, wherein the antibody is anti-phospholipase A₂, anti-CD14 or anti-Toll 4 receptor antibody.

3. The composition of claim 1, wherein the fish food comprises synthetic or plant-derived proteins.

4. The compositions of claim 1, wherein the fish food comprises synthetic or plant-derived lipids.

5. The composition of claim 1, wherein the fish food comprises fat and protein.

6. The composition of claim 5, wherein the composition comprises by weight from 30% to 65% protein and from 5% to 25% fat.

7. The composition of claim 1, wherein an egg preparation comprises the antibody.

8. The composition of claim 7, wherein the composition comprises fom about 0.05% to about 2% by weight of the egg preparation.

9. The composition of claim 1, further comprising a vegetable oil coating.

10. Thie composition of claim 9, wherein the vegetable oil coating comprises from about 1% to about 5% vegetable oil.

11. A method comprising administering to fish or shellfish an antibody that reduces the bioavailability of a prostaglandin or leukotriene lipid precursor in an amount effective to increase feed efficiency and/or weight gain compared to fish or shellfish which have not been administered the antibody.

12. The method of claim 11, wherein the antibody is administered as a composition comprising fish food.

13. The method of claim 11, wherein the antibody is administered to a fish selected from trout, tilapia, perch, catfish, salmon, bass, sunfish, and baitfish.

14. The method of claim 11, wherein the antibody is administered to a shellfish selected from shrimp, prawns, mussels, clams, oysters, lobster, crab, and crayfish.

15. The method of claim 11, wherein the weight gain of the fish or shellfish is at least 10% relative to fish or shellfish not administered the composition.

16. The method of claim 11, wherein the antibody is an anti-phospholipase A₂, anti-CD14 or anti-Toll 4 receptor antibody.

17. The method of claim 11, wherein the feed efficiency is increased at least 5% relative to fish or shellfish not administered the composition.

18. A method comprising administering to a fish or a shellfish an antibody that enhances weight gain by the fish or shellfish by at least 10%, improves feeding efficiency by at least 5%, or both compared to untreated fish.

19. The method of claim 18, wherein the antibody is an anti-phospholipase A2, anti-CD14 or anti-Toll 4 receptor antibody.

20. The method of claim 18, wherein the antibody is administered by feeding it to the fish or shellfish in food.
